(19) United States
(12) Patent Application Publication
(10) Pub. No.: US 2016/0281045 A1
(43) Pub. Date: Sep. 29, 2016

(54) Cell Culture System

(71) Applicant: LOUGHBOROUGH UNIVERSITY, Loughborough, Leicestershire (GB)

(72) Inventors: Mark MCCALL, Loughborough, Leicestershire (GB); Robert THOMAS, Loughborough, Leicestershire (GB)

(21) Appl. No.: 15/036,948

(22) PCT Filed: Nov. 14, 2014
(86) PCT No.: PCT/GB2014/053379

§ 371 Date: May 16, 2016

(30) Foreign Application Priority Data
Nov. 15, 2013 (GB) 1320174.4

(51) Int. Cl. C12M 1/12; C12M 1/00; C12N 5/0775; C12N 5/00

(52) U.S. Cl. CPC C12M 25/18; C12N 5/0075; C12M 23/40; C12M 23/34; C12N 5/0668

(57) ABSTRACT

The present invention provides methods, devices and systems of culturing cells in a cell culture5 device comprising a plurality of non-porous substrates in the form of a packed bed in which the addition of substrates to the cell culture device enables specific yields to be obtained. Modified non-porous substrates are also provided for use in such methods, devices and systems.

### FIELD OF THE INVENTION

The present invention relates to a scalable packed-bed cell culture device and methods of using thereof.

### BACKGROUND OF THE INVENTION

Cellular (cell) therapy can be defined as the use of cells to treat disease. Ex vivo expansion of cells obtained from human donors is being used, for example, to increase the numbers of stem and progenitor cells available for autologous and allogeneic cell therapy. Ex vivo culturing of cells may also provide materials necessary for research in pharmacology, physiology, and toxicology.

As an example of this practice multipotent mesenchymal stem cells (MSCs) are currently exploited in numerous clinical trials to investigate their potential in, amongst other uses, tissue regeneration and immune regulation. The relatively low frequency of MSCs in all clinically relevant donor material necessitates cell expansion to achieve significant transplant doses.

The challenge for any cell therapy manufacturer is to assure safe and high-quality cell production. In particular, cell processing under Good Manufacturing Practice (GMP) is mandatory for the progress of such expanded cell therapies. For all cell therapies that include an expansion or manipulation step, the economics of testing and certification of processes and products for GMP compliance are a significant cost factor in cell manufacturing, strongly encouraging production of maximum batch sizes and minimum batch run.

Large-scale cell culture expansion processes and technology have been deployed extensively over years for the growth of bacteria, yeast and moulds. These microbial cells all possess robust cell walls or extra cellular matrices that make them less sensitive to variations in culture conditions. The structural resilience of these microbial cells is a key factor allowing cost effective and rapid development of highly-efficient cell culture processes for these types of cells. For example, bacterial cells can be grown in very large volumes of liquid medium using vigorous agitation, culture stirring and gas sparging techniques to achieve good aeration during growth while maintaining viable cultures. In contrast, techniques used to culture cells such as eukaryotic cells, animal cells; mammalian cells and specifically clinically relevant human cells are more difficult and complex because these cells are relatively delicate. These cells can be easily damaged by excessive shear forces, a result of vigorous agitation and aeration that is necessary to maintain microbial culture in conventional bioreactor/fermentation systems.

Large-scale automated, closed processes for use of mammalian cells to manufacture proteins, such as biotherapeutics, are well established. However, most such processes are designed to recover a protein product and discard the cells under conditions leading to cell death. In contrast, processing of therapeutic cells after expansion typically requires cell harvesting. As a result these systems are not optimized to provide a large expansion ratio—where expansion ratio is defined as the output cell population divided by the input cell population. Processes requiring more process steps (transferring from one vessel to another) to achieve a given overall expansion ratio will require more manipulation of the cell population—resulting in higher costs and potentially lower quality cells.

Accordingly, there is a need for improved processes for manufacturing therapeutic cells that minimize production costs and maximize process expansion ratios.

### DESCRIPTION OF PRIOR ART

A general example of a basic cell-cultivating system is the manual or automated manipulations of tissue flasks. Manual use of tissue flasks is a well-accepted method of researching and developing cell therapy manufacturing processes. The most developed of these technologies can only provide a surface area for cell adhesion of around ˜1750 cm². For large scale, manufacturing of therapeutic cell types, hundreds or thousands of tissue flasks would need to be simultaneously taken care of in a factory scale up setting, requiring a great deal of labour. Implementing automated manipulation of tissue flask cell-cultivation can save labour, but is highly capital intensive and time consuming.

Another widely researched example of cell-cultivating systems is a stirred tank fermenter or bioreactor. The bioreactor will usually employ microcarriers inside to provide a surface area for cells to adhere to—although some now propose using cell aggregates. While this provides the opportunity to scale the culture process, stirring culture medium and gassing can considerably affect the metabolic activity or quality of the cells. Operation conditions may need to be changed when the dimensions of the stirring tank are enlarged. Changes of the operation conditions greatly delay the product development as more validation of the output cell quality is required.

A further example of a cell cultivating system is hollow fibre cartridge based bioreactors. Within this system cell density can reach 10⁸ per ml in the bioreactor extra capillary space. This reactor vessel faces a significant limitation—when the cell density increases towards its maximum level the cells at the rear end of the bioreactor cannot obtain enough nutrition or oxygen and cell expansion will be inhibited. Consistent, repeatable recovery of cells from the extracapillary space is challenging due to the cell inhibiting fluid flow. To avoid such a situation, the reactor generally is not made large, which is a major disadvantage of hollow fibre reactor designs.

A final and relatively underdeveloped area of cell therapy manufacturing platforms is packed-bed bioreactors. These typically contain porous matrixes that provide a high area for cell growth and protect cells from shear forces. A relatively high density of over 5×10⁷ mammalian cells/ml been reported. However within these fixed high density matrixes fluid flow is not homogeneous. Medium flows with greater ease through local regions of low packing density and have reduced or impeded flow in regions with higher packing density. Despite attempts to develop homogenous matrixes, uneven cell seeding and expansion within these packed beds still create a heterogeneous local microenvironment around the cell population. These are variation of a channelling effect. The channelling effect impedes cell growth and causes cell death in those regions with high packing density as media flow is cut off. Regions of high cell density also suffer reduced cell recovery leading to inconsistent cell harvests. Therefore, eliminating the nutrient/oxygen gradient, the channelling effect, and improving fluid flow distribution are key factors in unlocking the scale limitation of a packed-bed bioreactor system for cell therapies.

A further limitation common to tissue flasks, hollow fibre bioreactors and packed bed bioreactors is the inability to non-destructively sample the cell population within the system for in process population monitoring—a key requirement of stem cell cultivation. This limits the ability of cell therapy developers to optimize expansion processes during development.

More conventional designs of packed-bed culture device, such as U.S. Pat. No. 5,501,971, issued to Freedman et al, discloses a method and apparatus for cultivating cells in a reactor that includes a basket-type packed bed and an internal liquid cell growth medium recirculation device consisting of a stirrer. This design will have all above mentioned drawbacks such as nutrient gradient, media channelling effects and a heterogeneous distribution of cells that limits the practical bed scale to below 10L. This system also uses a comparatively high level of media per surface area when compared with hollow fibre cartridges for example as the basket sits within a bath of media.

Recently inventions have tried to overcome the scale-up limitations inherent within current packed-bed systems. U.S. Pat. No. 5,766,949, issued to Liau et al. describes a cell-cultivating system in which the culture medium oscillates up and down with respect to a growth substrate in an attempt to improve the oxygenation of the cells.

Liau's design however, presents many disadvantages. One disadvantage of this system is the complexity of Liau's apparatus. The Liau system requires two external storage tanks and a separate growth chamber which holds the substrate. Multiple peristaltic pumps are required to circulate the growth medium from one storage tank through the culture chamber and then into another storage tank and then back to the first storage tank. Sterilization is potentially difficult and laborious due to a relatively large amount of components to the apparatus and the size of apparatus. In addition, due to the complexity of the system, the harvesting of the cell population or any secreted protein or cellular product would be cumbersome and time consuming. Lastly, when the growth medium is lowered with respect to the growth substrate plates, the cells become exposed to air, i.e., gaseous environment directly, and thus, may result in cell death—particularly for delicate mammalian cells that may be used in cell therapies.

These packed bed, hollow fibre and tissue flask systems and stirred tank/microcarrier systems all suffer from a common limiting factor that the present invention seeks to remedy. All lack the ability of effectively regulate and control the cell spacing (local cell density) of adherent cells within the system apart from changing the initial seeding density and the point of harvest—between these points, the cell density is uncontrolled and often unmeasured. This property, local cell density, is critical in regulating the growth rate of cell populations and the cell secreted molecules (that both support and inhibit cell growth) that surround the cell population. Regulation of these secondary proprieties allow for example increased cell expansion ratio's, directed stem cell lineage and function, general cell health and vitality—all of which are attributes that are desirable to control effectively.

The present invention provides the ability to regulate and control the cell spacing (local cell density) of adherent cells within the cell culture system by the controlled addition and/or subtraction (and mixing) of substrates of the invention in a packed bed culture system.

Given the importance of cell and tissue culture technology in biotechnology research, pharmaceutical research, academic research, cell therapy manufacturing and in view of the deficiencies, obstacles and limitations exist in the prior art described the present invention overcomes the obstacle and remedies the deficiencies in the prior art by teaching and disclosing a method and an apparatus for cell and tissue culturing that fulfils the long-felt need for a novel method and apparatus to culture cells and tissues that provides a higher degree of cell environment and growth control in a relatively less complex, efficient, device capable of increasing the economies of production scale and potentially producing a higher yield of cellular by-products generated from the cells.

It will be easily understood by the skilled in the art that cells on carriers or microcarriers may have the ability to move from one carrier to another. Indeed it is well known that this is a key ability efficient cell culture. However this property is uncontrolled within stirred tank reaction systems as the microcarriers are inconstant movement in relation to each other and cells must transfer via floating in the culture medium which is again uncontrolled and potentially damaging for cell viability.

### SUMMARY OF THE INVENTION

The present invention provides many useful aspects and embodiments as described herein.

According to a first aspect of the invention, there is provided a method of culturing cells in a cell culture device comprising a plurality of non-porous substrates in the form of a packed bed, a reservoir for cell culture media in fluid communication with the cell culture device, and a means for circulating the cell culture media from the reservoir through said device, in which the method comprises:

(i) incubating the cells,

(ii) recirculating the media between the reservoir and the packed bed chamber,

(iii) introducing further substrates to the packed bed container once the cells have reached 40-50% confluence,

(iv) repeating step (iii) and adding additional substrates at a ratio of 20% to 60% extra surface area once the cells have reached 40-50% confluence,

(v) optionally repeating step (iv)

(vi) comprising a step of mixing substrates in the device,

(vii) optionally sampling the cell culture to measure the cell density, and

(viii) recovering the cells.

The cells may be introduced to the substrates in the cell culture device, mixed with the substrates and both introduced together into the cell culture device, or the substrates may be added to the cells in the cell culture device.

In step (iii) the cells may reach a confluence of from 40% to 50%, suitably from 45% to 50%, or from 40% to 45%. The desired confluence may depend in part on the cells being cultured and the conditions of the culture. The additional substrates in step (iii) may be added at a ratio of from 20% to 60% extra surface area for each further day of culture, suitably from 30% to 50% extra surface area for each further day of culture, suitably of from 35% to 45%, 30% to 40%, 35% to 50%, or 45% to 50%. Suitable addition ratios may be 30%, 35%, 40%, 45%, or 50%. The amount of additional cell culture substrates may also be changed according to the required output of the system in operation for each further day of culture.

In step (iv), the additional cell culture substrates may be added at a ratio of from 20% to 60% extra surface area for each further day of culture, or from 30% to 50% extra surface area for each further day of culture, suitably of from 35% to 45%, 30% to 40%, 35% to 50%, or 45% to 50%. Suitable addition ratios may be 30%, 35%, 40%, 45%, or 50%. The amount of additional cell culture substrates may also be changed according to the required output of the system in operation for each further day of culture.

The cell culture substrates are suitably rigid and may comprise a tubular section of an inert material having an external surface and an internal surface which defines a lumen having a polygonal or circular cross section in which the maximum distance across the lumen is approximately the same as the vertical height of the substrate. The substrates also suitably have regular and uniform dimensions, i.e. the substrates are homologous or identical which provides for consistently random packing arrangements of the substrates in the cell culture device. The substrates may therefore suitably be randomly packed in the packed bed.

Step (iv) may be optionally further repeated to include additional substrate for each additional day of culture.

The method of culturing cells in accordance with the present invention may provide for an expansion ratio in the number of cells present in the culture of at least 50-fold, at least 75-fold, at least 100-fold, at least 140-fold, at least 150-fold, at least 160-fold, at least 170-fold, or at least 175-fold, at least 180-fold, at least 190-fold, at least 200-fold, at least 250-fold, at least 300-fold, or higher after introduction of further substrates in step (iii) and additional substrates in step (iv) of the method.

The method may be performed for a number of days of culture, suitably for at least one day or at least two days, for 1 to 3 days, for 1 to 4 days, for 1 to 5 days, for 1 to 6 days, 1 to 7 days, 1 to 8 days, 1 to 9 days or 1 to 10 days or longer. The method may be carried out for as long as the cells are dividing so for an immortalised cell line the method can be performed for as long as is required.

The non-porous substrates typically have external dimensions of between about 1.0 mm to about 10.0 mm. Suitably, the substrates typically have a shape that allows for consistent random packing within the packed bed container (such as Raschig rings or modified cell culture substrates as described herein). The substrate shape may include a channel, plurality of channels or other feature that allows cell culture media to pass freely within the substrate conduit.

The substrates may be constructed of any material, or coated with any material that promotes the attachment and growth of adherent cell populations. Suitably, the substrates may be composed of non-expanded polystyrene or any rigid polymer that is biologically compatible, glass or ceramic. The substrates may be formed by any available commercial process such as extrusion or batch injection molded. The substrates may be coated with a biologically active substance, for example a hormone, a growth factor, matrix protein or a mixture thereof.

The terms “substrates” and “micro-substrates” may be used interchangeably. As used herein the term “passage” of cells refers to culture of cells wherein the cells remain viable but may or may not be actively dividing. Furthermore the term “expansion” refers to growth of dividing cells wherein the number of cells increases with culture time.

The cells may be cultured in the device according to the culture conditions desired in order to culture the cells present. Such protocols are well-know and established according to the nature of the cells being cultured.

The cell culture device may be open or closed as required. Suitably the device will be sterile in operation and suitable for operation under aseptic conditions. The device may be sterilised by any suitable procedure, such as for example steam sterilisation, radiation etc.

The cell culture media will be appropriate to the cells being cultured and the desired end-point for the culture. For example, if growth of the cells is required a growth medium will be used. For differentiation of undifferentiated cells a suitable differential medium or media will be used as required (if the differentiation protocol requires different media to be used at different times in the period of culture.

The means for circulating the cell culture media from the reservoir through said device may be a pump, or other suitable means to direct the flow of media in a continuous direction through the device.

The mechanism of mixing in step (vi) may take the form of a mixing arm, impeller, lever etc. that resides within the packed bed container. The mechanism of mixing may take the form of controlled liquid flow such as perfusion or stirring of the media within the packed bed container. The mechanism of mixing may take the form of a magnetic force applied to micro-substrates within the packed bed container. The mechanism of mixing may take the form of altering the physical orientation of the packed bed chamber by moving, shaking, turning, rotating etc.

The cell density may be measured by sampling at step (vii), or alternatively the cell density may be measured by sampling the cell culture to measure the cell density after recovering the cells.

The method therefore permits process scaling in view of the addition of substrate to the expanding flexible packed bed of the device. The bed volume can be increased as required in a method of the invention, or alternatively where culturing an immortalised cell line, the volume of cell substrates can be halved every day and then fresh substrate can be added for a continuous culture of the same volume of substrates in the packed bed. The invention therefore provides for a method of continuous cell culture.

In one embodiment of the invention, there is provided a method of culturing cells in a cell culture device comprising a plurality of substrates in the form of a packed bed, a reservoir for cell culture media in fluid communication with the cell culture device, and a means for circulating the cell culture media from the reservoir through said device, in which the method comprises:

(i) incubating the cells,

(ii) recirculating the media between the reservoir and the packed bed chamber,

(iii) introducing further substrates to the packed bed container once the cells have reached 40-50% confluence,

(iv) optionally repeating step (iv) and adding additional substrates at a ratio of 30% to 50% extra surface area for each further day of culture,

(v) optionally sampling the cell culture to measure the cell density, and

(vi) recovering the cells.

In another alternative embodiment of the invention, the method comprises:

(i) incubating the cells,

(ii) recirculating the media between the reservoir and the packed bed chamber,

(iii) introducing further substrates to the packed bed container once the cells have reached 40-50% confluence,

(iv) repeating step (iii) and adding additional substrates at a ratio of 30% to 50% extra surface area for each further day of culture, and

(v) recovering the cells.

The packed bed in accordance with the present invention is an expanding bed cell culture. In other words the packed bed is not a fixed bed or rigid bed. The bed increases in volume over the lifetime of the cell culture process. The bed is therefore a flexible or a loose-packed bed. The invention therefore provides a method for the culturing of cells on a flexible or a loose-packed bed.

An advantage of the present invention is that it permits the sampling of the cell culture without disruption to the packed bed. In prior art fixed bed cultures which are rigid, the step of sampling the cells leaves a hole which encourages media flow through the hole thus compromising the efficiency of the culture process. In the method of the present invention, the sampling of the flexible bed also leaves a void but this is rapidly filled-in and the bed heals itself.

The invention provides for the frequency and quantity of the addition of substrate to be varied in order to produce a specific yield from a cell culture.

According to a second aspect of the present invention there is provided a cell-culture device comprising a packed bed chamber and a control unit, in which the control unit comprises a means for monitoring one or more process parameters, wherein the packed bed chamber comprises a plurality of openings for introducing or removing non-porous substrates and/or cells and permitting flow of cell culture media into and out of the device.

The packed bed in accordance with the present invention is an expanding bed cell culture. In other words the packed bed is not a fixed bed or rigid bed. The bed increases in volume over the lifetime of the cell culture process. The bed is therefore a flexible or a loose-packed bed. The invention therefore provides a device for the culturing of cells on a flexible or a loose-packed bed. As described above, the substrates may suitably be randomly packed in the packed bed.

The device therefore permits process scaling in view of the addition of substrate to the expanding flexible packed bed of the device. The bed volume can be increased as required in a method of the invention, or alternatively where culturing an immortalised cell line, the volume of cell substrates can be halved every day and then fresh substrate can be added for a continuous culture of the same volume of substrates in the packed bed. The invention therefore provides for continuous cell culture.

The cell culture device may be open or closed as required. Suitably the device will be sterile in operation and suitable for operation under aseptic conditions. The device may be sterilised by any suitable procedure, such as for example steam sterilisation, radiation etc.

The device may suitably comprise a means for circulating the cell culture media from a reservoir through said device which may be a pump, or other suitable means to direct the flow of media in a continuous direction through the device.

The mechanism of mixing the substrates in the device may take the form of a mixing arm, impeller, lever etc. that resides within the packed bed container. The mechanism of mixing may take the form of controlled liquid flow such as perfusion or stirring of the media within the packed bed container. The mechanism of mixing may take the form of a magnetic force applied to micro-substrates within the packed bed container. The mechanism of mixing may take the form of altering the physical orientation of the packed bed chamber by moving, shaking, turning, rotating etc.

According to a third aspect of the invention there is provided a system for culturing cells comprising a cell culture device of the second aspect of the invention, a reservoir for cell culture media in fluid communication to the cell culture device, a means for circulating the cell culture media from the reservoir through said device, and a reservoir of cell culture non-porous substrates in connection to the cell culture device and a means for introducing said substrates from the reservoir to the cell culture device.

In an embodiment of the invention there is provided a system for culturing cells comprising a cell culture device containing a plurality of non-porous substrates, a reservoir for cell culture media in fluid communication to the cell culture device, a means for circulating the cell culture media from the reservoir through said device, and a reservoir of non-porous cell culture substrates in connection to the cell culture device and a means for introducing said substrates from the reservoir to the cell culture device.

Systems of the invention as defined above may comprise substrates having a lumen with a circular cross section in which the diameter of the lumen is approximately the same as the vertical height of the substrate or in which the cell culture substrate may be as defined herein below.

Features of the cell culture device of the second aspect of the invention and the system of the third aspect of the invention are as defined above in relation to the method of the first aspect of the invention mutatis mutandis.

The present invention therefore provides a system and method of operation of a packed bed cell culture device with the objective of regulating the adherent cell density within the packed bed.

In one embodiment, there is provided a system for the controlled maintenance and expansion of adherent cells, comprising:

(a) a packed bed comprising a cell culture device having an outlet and an inlet and containing therein a non-rigid three dimensional non-porous substrate comprising of a plurality of smaller discrete non-porous substrates (micro-substrates) according to the present invention. The non-porous substrates of the invention (micro-substrates) are suitably capable of supporting the attachment and growth of adherent cell populations.

(b) a mechanism for the controlled addition/subtraction of the aforementioned micro-substrates with the objective of producing any desired ratio of cell density to surface area of micro-substrates designated as CD/SA between a maximum, corresponding to the maximum surface density of the adherent cell population achievable, and essentially zero. A secondary objective of producing any desired rate of cell growth on the available surface area between a maximum, corresponding to the maximum growth rate of the cell population, and essentially zero. A tertiary objective of regulating the relative local densities of cells and cell secreted factors such as, but not limited to, cytokines, proteins and extracellular matrix. These three objectives intended to produce consistent large scale culture of adherent cell populations by enabling a large expansion ratio and improving cell viability.

This mechanism of addition may take the form of an additional chamber attached to the packing container that contains a new supply of micro-substrates by means of a sterile joint, connection, weld or other communications means such as a tube, pipe or channel.

This mechanism of addition may take the form of a chamber (or plurality of chambers) within the packed bed chamber that contain a new supply of non-porous micro-substrates that may be added to the micro substrates by the removal of a dividing feature or a change in physical orientation of the packed bed container.

(c) a mechanism for mixing the non-rigid arrangement of micro-substrates within the substrate to distribute newly added micro-substrates within the substrate to support the objective of producing any desired ratio of cell density to surface area of micro-substrates designated as CD/SA between a maximum, corresponding to the maximum surface density of the adherent cell population achievable, and essentially zero. Said mechanism also supporting a secondary objective of producing any desired rate of cell growth on the available surface area between a maximum, corresponding to the maximum growth rate of the cell population, and essentially zero and the tertiary objective of regulating the relative local densities of cells and cell secreted factors such as, but not limited to, cytokines, proteins and extracellular matrix that may inhibit or support cell growth. These three objectives intended to produce consistent large scale culture of adherent cell populations by enabling a large expansion ratio and improving cell viability.

(d) a secondary unit, connected to the packed bed chamber by a media communications means, that includes mechanisms of controlling the state of the cell culture media (pH, dissolved oxygen, temperature, glucose level as examples) that is passed from this unit (called the controlling unit) to the packed bed container by the communications means etc.

The systems of the present invention may also be configured so that the controlling chamber(s), addition and mixing mechanisms and packed bed container may all be contained within one single object/unit.

The present invention provides a range of methods of culturing adherent cells within the cell culture device of the invention, which may be a packed bed container—each different method comprising of a mixture of the following scaling activities.

A first method may comprise seeding adherent cells onto the micro-carriers as mentioned above prior to incorporation into the packed-bed container. A second method may comprise injection or infusion of a cell inoculum into the packed bed chamber prior to the commencement of culture.

The adherent cells can be maintained by the continuous or pulsed flow of cell culture media within the packed bed container.

Cell density within the packed bed can be maintained by the controlled addition or removal of new or used micro-substrates either continuously or at prescribed intervals. The new micro-substrates can be mixed into the bed as described above.

The timed removal or sampling of micro-substrates with cells attached can be performed for the monitoring of cell health and density.

The harvesting of cells from within the packed bed compartment can be achieved by infusion of an enzymatic treatment or equivalent.

The present invention eliminates the limitation of current packed bed technology by providing active control of cell surface density and environment within the bed.

The present invention enables scaling up of bench-scale processes to larger economic volumes whilst achieving a highly controlled cell density, environment and cell yield.

The following detailed description, given by way of example, is not intended to limit the invention to any specific embodiments described. The detailed description may be understood in conjunction with the accompanying figures. Without wishing to unnecessarily limit the foregoing, the following shall discuss the invention in greater detail.

The embodiments of the present invention can be used to culture any cells requiring an attachment substrate. The embodiments of the present invention can be used to produce any products generated from such cells, such as recombinant proteins, enzymes and/or viruses.

In one embodiment of the present invention, the cell-cultivating device contains two primary chambers: a packed bed container and a controlling unit. The controlling unit attached to a computer for monitoring and control of the process parameters. The packed bed chamber comprises a plurality of openings for introducing or removing micro-substrates and providing media flow. A micro substrate mixing means is installed inside or outside the packed bed container. For the mixing means inside the packed bed chamber, a liquid flow manifold is preferred; for outside the packed bed container, a shaker, rocker, rotating arm is preferred. The packed bed container is preferably disposable, and of course it could be a rigid metal, glass or plastic container as well. A media distribution manifold is preferably installed inside the packed bed container optionally to enhance even media distribution. At least a tube is used for communicating the packed bed container and the control vessel. The control vessel is supported in a platform that could provide temperature control and mixing to homogenize the culture medium inside the control vessel. The control and packed bed containers can also be installed with pH, DO or temperature probe for monitoring and process control.

In another embodiment the packed bed chamber may also contain an impeller or integrated pump for recirculation of the media within the packed bed container.

In a further embodiment the packed bed container may also be divided into separate compartments to segregate micro-substrates.

The embodiments may all employ either a method that uses recirculating media flow from the control vessel to the packed bed container or a single flow from the control vessel to the packed bed chamber to a third chamber such as a harvest vessel or waste container.

In an alternative embodiment the non-porous micro-substrates may take the form of small hollow cylinders or alternatively small Raschig rings.

Methods, systems and cell-culture devices of the invention may be used to culture cells of any type. The invention may find particular application in the culture of animal cells, especially mammalian cells (human or non-human-cells), for example primate cells (e.g. human cells), rodent cells (e.g. murine cells) etc. It is envisaged that any animal cell type or origin may be cultured in accordance with the various aspects and embodiments of the present invention, including somatic cells and stem cells. Stem cells may include induced pluripotent stem (iPS) cells, mesenchymal stem cells (i.e. adult stem cells, including haematopoietic stem cells), and embryonic stem (ES) cells. Stem cells or progenitor cells (stem-like cells) from any animal tissue may therefore be cultured accordingly.

Another advantage of the method of invention is that it avoids the need for a separate passage step and permits continuous culture in a single device to occur. The invention permits the control of the localised cell density within the packed bed regardless of the total bed volume and duration of the cell culture process.

The present invention also provides for modified cell culture substrates as an alternative to standard Raschig rings. The modified Raschig rings may be used in any of the methods, systems and cell-culture devices of the invention.

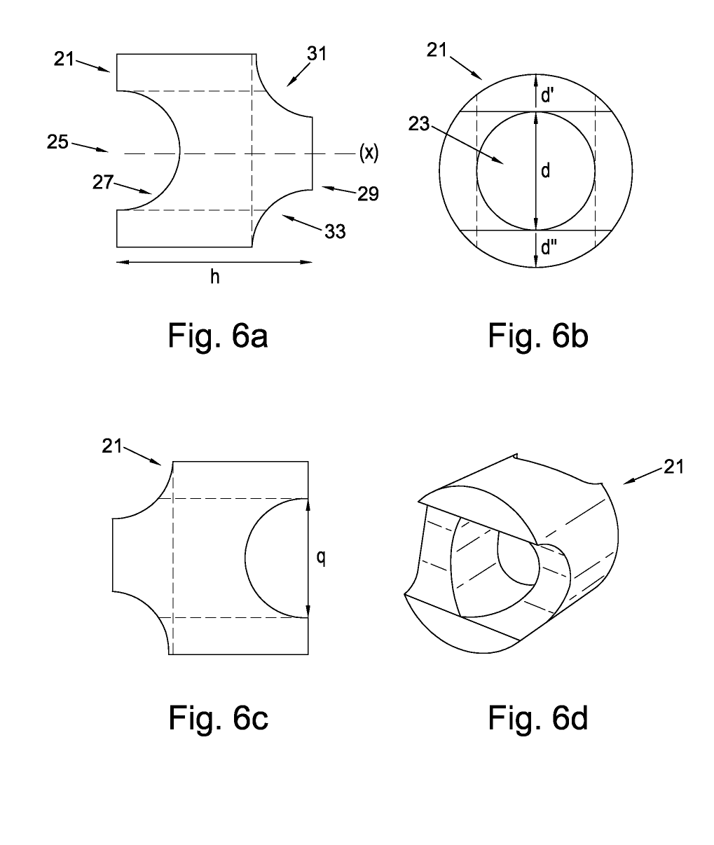

One form of the modified cell culture substrate comprises a tubular section of an inert material with a circular cross section, which has an interior lumen having a circular cross section in which the diameter of the lumen is approximately equal to half the diameter of the substrate, in which the vertical height of the substrate is approximately 1.5 times the diameter of the substrate and in which one end of the substrate has a channel cut perpendicular to the axis of symmetry of the lumen where said channel has a diameter approximately equal to the diameter of the lumen of the substrate. The cell culture substrate may be further modified at the other end by the presence of one more channels. One embodiment is as shown in FIG. 6.

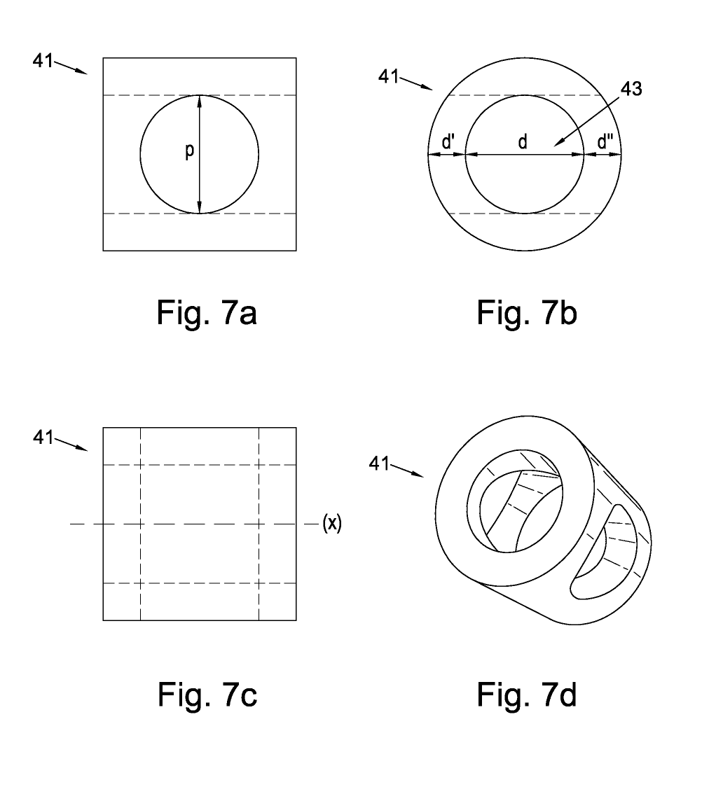

Alternatively, the cell culture substrate may comprise a channel cut through the substrate at the centre of the substrate. One embodiment is as shown in FIG. 7.

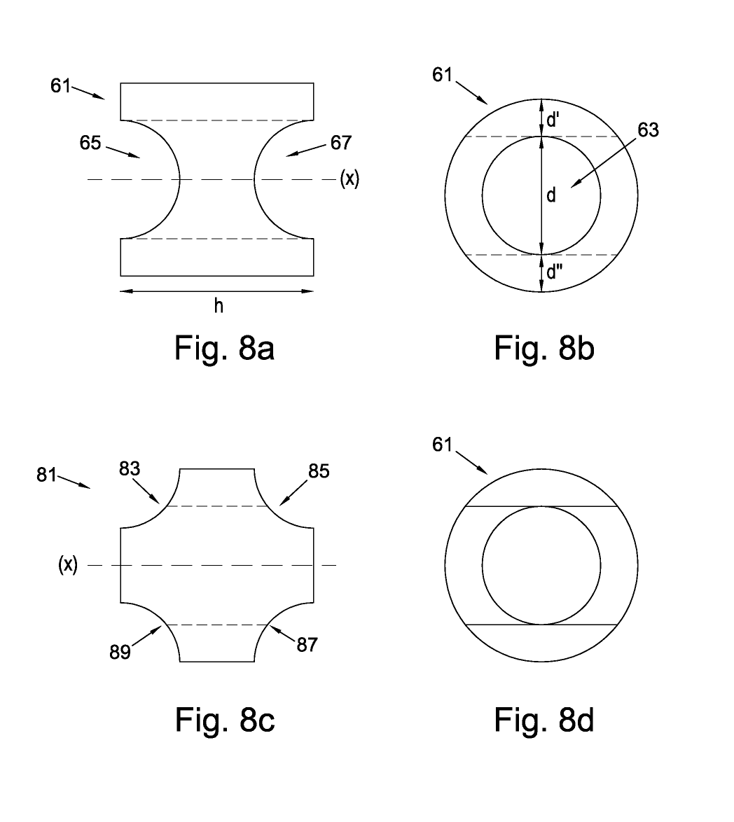

Another modified form of the cell culture substrate may have a channel cut through the substrate at both ends of the substrate. One embodiment is as shown in FIG. 8.

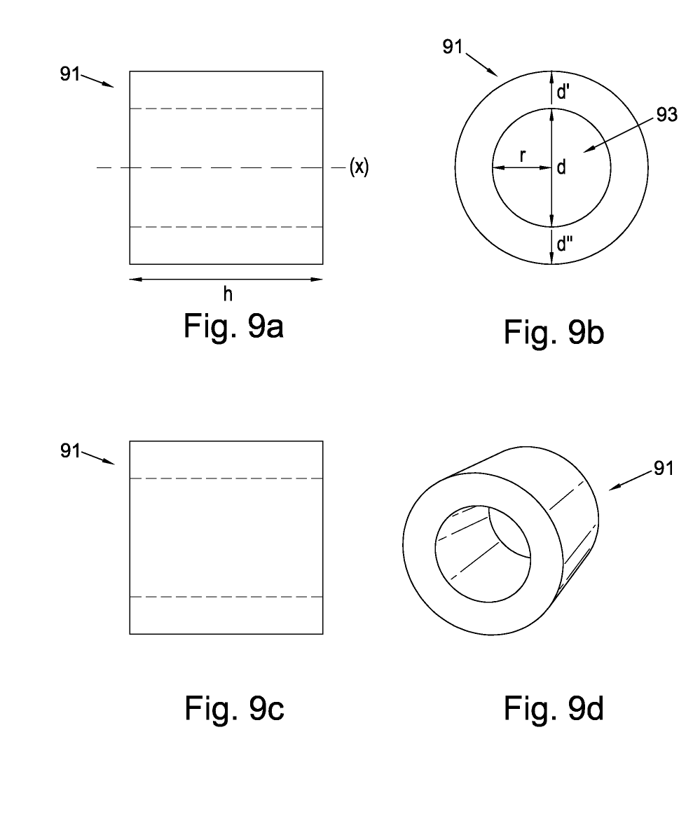

The cell culture substrate may also comprise a tubular section of an inert material with a circular cross section, which has an interior lumen having a circular cross section, in which the vertical height of the substrate is approximately 1.5 times the diameter of the substrate and in which the radius of the lumen is approximately equal to the thickness of the wall of the substrate surrounding the lumen. One embodiment is as shown in FIG. 9.

Any of the cell culture substrates used in accordance with the present invention or as described above may be coated with a biologically active substance, for example a hormone, a growth factor, matrix protein or a mixture thereof.

The height of the cell culture substrate may be from about 0.5 mm to about 10.0 mm, suitably about 1.0 mm to 9.5 mm or about 1.0 mm to about 5.0 mm, or 3.0 mm to about 8.5 mm, or 3.5 mm to 7.5 mm, suitably of from 3.0 mm to 4.0 mm. The height may selected from a range of values of 0.5 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm 8.0 mm, 8.5 mm, 9.0 mm, 9.5 mm and 10.0 mm.

The cell culture substrate may comprise a plurality of lumina (e.g. for example in order to achieve a honeycomb effect).

An advantage of the modified Raschig rings is that they provide a shorter path of any cells within the lumen to travel to reach a ‘new’ piece of surface area when added. This reduces the chance of localised areas of confluence forming inside the rings which a) aids in a homogenous cell distribution and b) improves the chances of cell transfer between rings. The same effect could be achieved by shortening the length of the Raschig rings (hence losing the Raschig property)—however the resulting packing would not mix as efficiently and would not pack as “randomly” as rings which retain a similar length and diameter.

As indicated above, preferred features for the second and subsequent aspects of the invention are as for the first aspect mutatis mutandis.

In the present application, reference is made to a number of drawings in which:

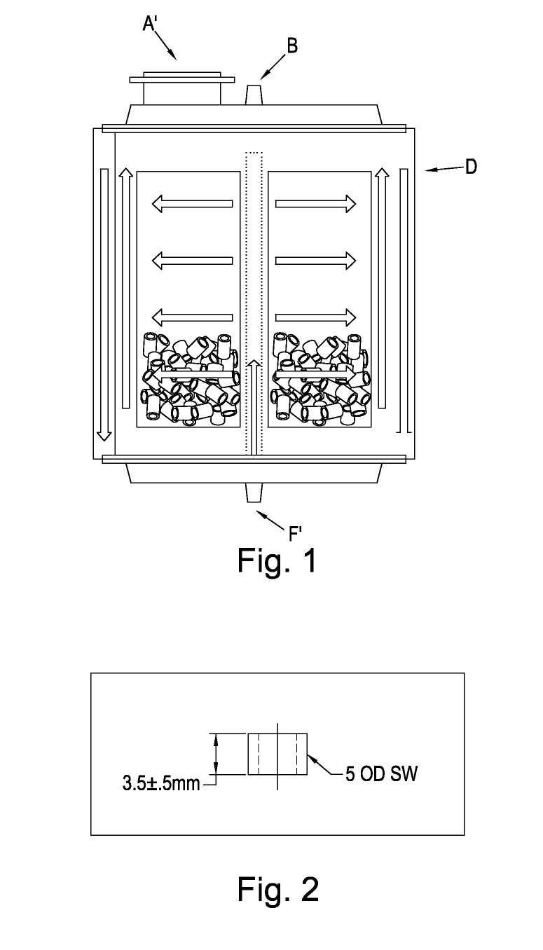

FIG. 1 shows one embodiment of the invention which employs either linear flow of media from one side of the packed bed container to another or the radial flow of the media from the centre of the packed bed container to the walls of the chamber or visa-versa. The packed bed container is shown in a vertical cross-section. The container as shown which comprises a vessel body (D), a media inlet and surface area addition inlet (A′), a sample inlet port (B), a media outlet and a sample outlet port (F′).

FIG. 2 shows one embodiment of the micro-substrates of the invention which are in the form of small hollow cylinders or alternatively small Raschig rings.

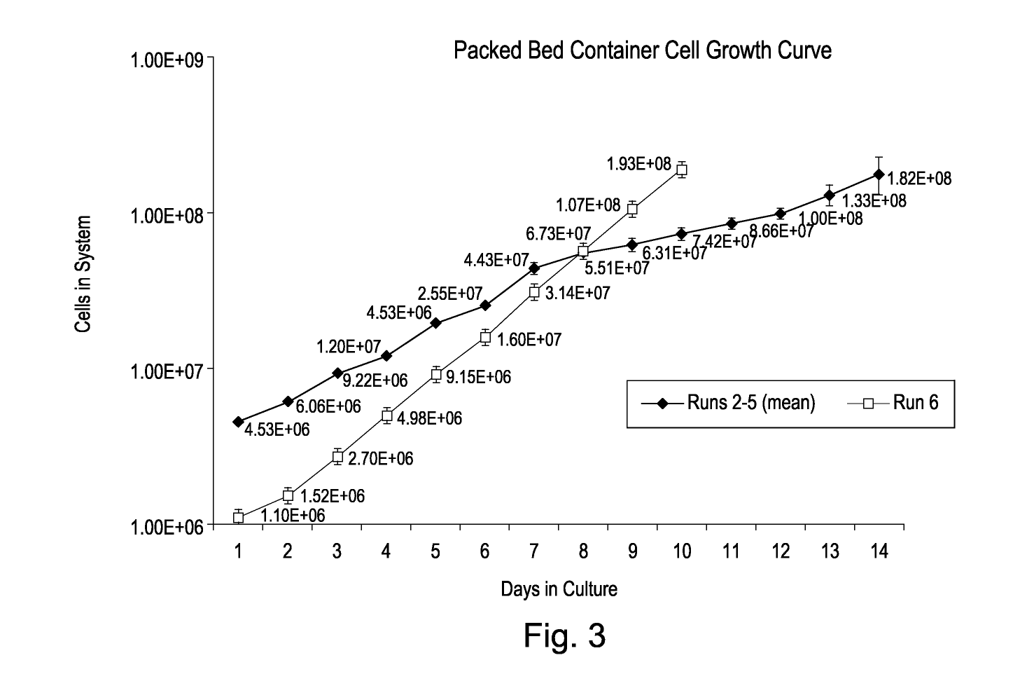

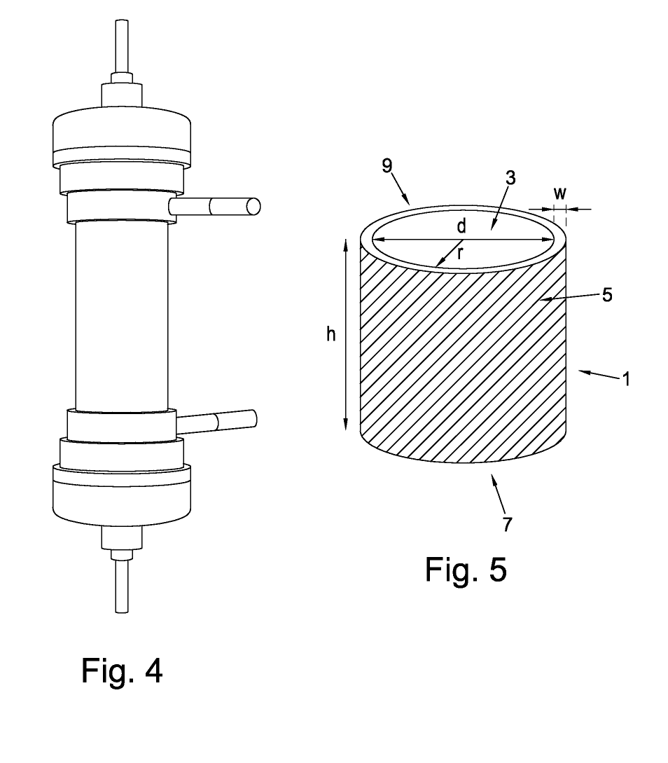

FIG. 3 shows a cell growth curve for a packed bed container of the invention.

FIG. 4 shows a cell culture device which may be used in accordance with the methods and systems of the invention. The device may be filled with cell culture substrates and cells with appropriate cell culture media, subjected to appropriate culture conditions as described herein.

FIG. 5 shows a perspective view of a simplified diagram of a Raschig ring.

FIG. 6 shows a modified Raschig ring of the invention (Packing configuration 1). FIG. 6a shows a side view, FIG. 6b shows a cross-sectional view, FIG. 6c shows the reverse side view, and 6d shows a perspective view.

FIG. 7 shows a modified Raschig ring of the invention (Packing configuration 2). FIG. 7a shows a side view, FIG. 7b shows a cross-sectional view, FIG. 7c shows the reverse side view rotated through 90°, and 7d shows a perspective view.

FIG. 8 shows a modified Raschig ring of the invention (Packing configuration 3). FIG. 8a shows a side view, FIG. 8b shows a cross-sectional view, FIG. 8c shows a reverse side view, and 8d shows a perspective view.

FIG. 9 shows a modified Raschig ring of the invention (Packing configuration 4). FIG. 9a shows a side view, FIG. 9b shows a cross-sectional view, FIG. 9c shows the reverse side view, and 9d shows a perspective view.

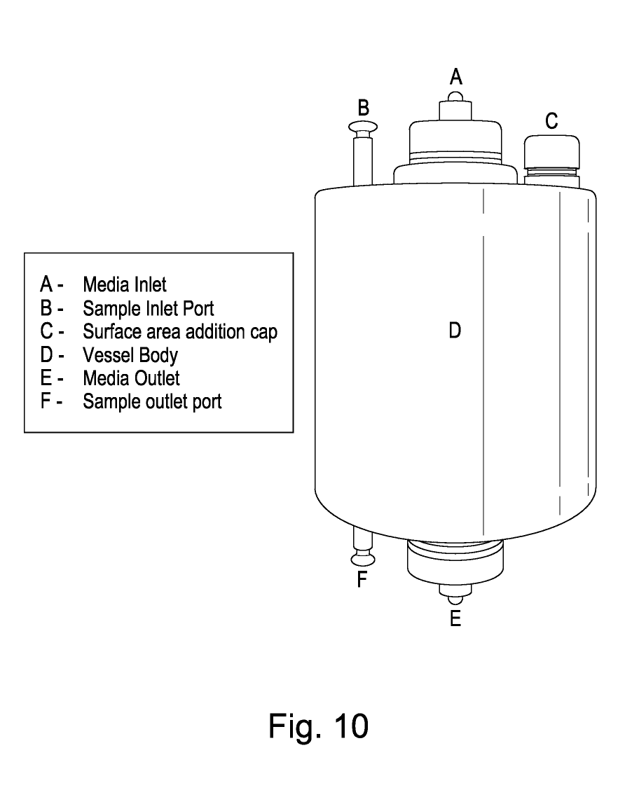

FIG. 10 shows a cell culture device of the invention.

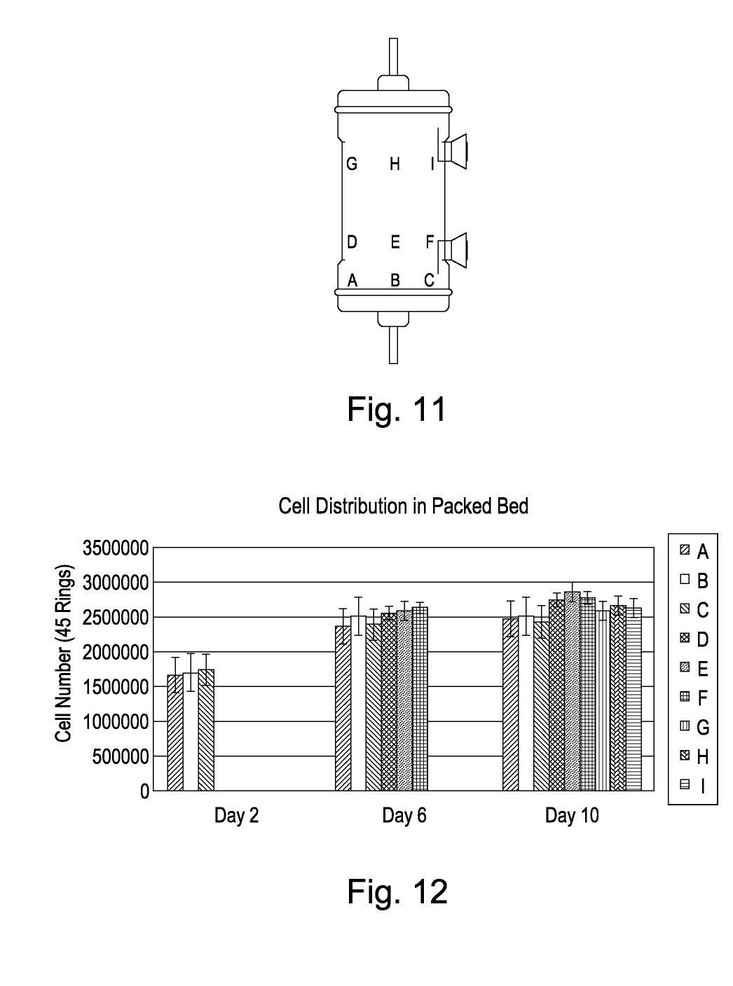

FIG. 11 shows sampling locations (A, B, C, D, E, F, G, H and I) within a packed bed vessel of the invention for examining the spatial distribution of cultured cell populations.

FIG. 12—shows results from spatial sampling of rings within a packed bed vessel of the invention. The results show an even distribution for cell distribution throughout the bed volume as it is increased over a ten day period.

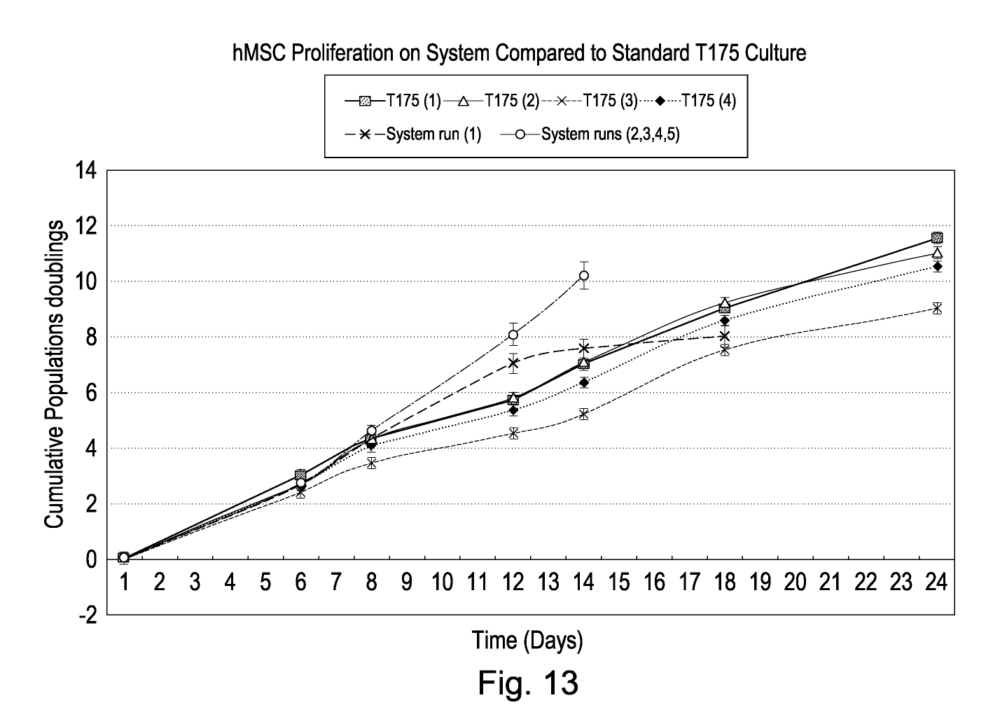

FIG. 13 shows a comparison of hMSC proliferation in a system of the invention compared to standard T175 cultures.

The present invention will now be further described by way of reference to the following Examples which are not to be construed as being limitations and are provided for the purposes of illustration only.

A standard Raschig ring which may be used as cell culture substrate in the methods, systems and cell culture devices of the invention is shown in FIG. 5. The Raschig ring (1) has a lumen (3) with a circular cross section in which the diameter (d) of the lumen is approximately the same as the vertical height (h) of the substrate. The Raschig ring is composed of a wall (5) which defines the lumen (3), having a first end (7) and a second end (9). The lumen (3) has a circular cross-sectional area defined by the diameter (d) and radius (r). The width (w) of the wall (5) is approximately 10% or less than the diameter (d). Suitably such rings are made of an inert material, such as plastic, glass, ceramic etc.

A modified cell culture substrate (21) of the invention is shown in FIG. 6. The substrate comprises a tubular section of an inert material with a circular cross section, which has an interior lumen (23) having a circular cross section in which the diameter of the lumen (d) is approximately equal to half the diameter (D) of the substrate (d+d′+d″), in which the vertical height (h) of the substrate is approximately 1.5 times the diameter of the substrate and in which one end (25) of the substrate has a channel (27) cut perpendicular to the axis of symmetry (x) of the lumen where said channel has a diameter (q) approximately equal to the diameter of the lumen of the substrate. The cell culture substrate also further modified at the other end (29) by the presence of two additional channels (31) and (33).

In an alternative embodiment (not shown), the channels (31) and (33) may be omitted.

A further modified cell culture substrate (41) of the invention is shown in Figure in which comprises a tubular section of an inert material with a circular cross section, which has an interior lumen (43) having a circular cross section in which the diameter (d) of the lumen is approximately equal to half the diameter (D) of the substrate (d+d′+d″), in which the vertical height (h) of the substrate is approximately 1.5 times the diameter of the substrate and in which a channel (45) is cut perpendicular to the axis of symmetry (x) of the lumen at the centre of the substrate where said channel has a diameter (p) approximately equal to the diameter (d) of the lumen of the substrate.

Another modified cell culture substrate (61) of the invention is shown in FIG. 8. The substrate comprises a tubular section of an inert material with a circular cross section, which has an interior lumen (63) having a circular cross section in which the diameter (d) of the lumen is approximately equal to half the diameter (D) of the substrate (d+d′+d″), in which the vertical height (h) of the substrate is approximately 1.5 times the diameter of the substrate and in which two channels (65, 67) are cut through the substrate at both ends of the substrate perpendicular to the axis of symmetry of the lumen where said channels have a diameter approximately equal to the diameter of the lumen of the substrate.

A variant is shown as modified cell culture substrate (81), in which further channels (83, 85, 87, 89) are cut into the substrate.

Another embodiment of the modified cell culture substrates of the invention is shown in FIG. 9. The modified cell culture substrate (91) comprising a tubular section of an inert material with a circular cross section, which has an interior lumen (93) having a circular cross section, in which the vertical height (h) of the substrate is approximately 1.5 times the diameter (D) of the substrate (d+d′+d″), and in which the radius (r) of the lumen is approximately equal to the thickness (d′ or d″) of the wall of the substrate surrounding the lumen.

A cell culture device of the invention is shown in FIG. 10, which comprises a vessel body (D), a media inlet (A), a sample inlet port (B), a surface area addition cap (C), a media outlet (E) and a sample outlet port (F).

### Example 1

### Human Mesenchymal Cell Culture Using the System

Procedure:

1. Micro-substrates were first seeded with 5000 cells per cm² on 100 of the micro-substrates as described in FIG. 2 and left to incubate for four hours to allow the cells to attach.

2. After incubation a recirculating media flow was established between the controlling vessel and the packed bed chamber. This was established via a peristaltic pump.

3. After 24 hours if the cells confluence had reached 40-50% then extra micro-substrates where added manually to the packed bed container and mixed in. The micro substrates in runs 2-5 as described in FIG. 3 where added at a ratio of 30% extra surface area each day of culture. In Run 6 they were added at a ratio of 50% extra surface area each day of culture.

4. Each day a sample of the micro-substrates where collected to sample the cell density.

5. After the packed bed chamber filled to capacity of micro substrates the cells where washed with a buffer solution and removed by enzymatic passage, allowing the recovery of the cells.

The relative growth curves are shown in FIG. 3 which demonstrates how changing the rate of surface area addition can significantly change the process results.

### Example 2

### Cell Distribution with a Packed Bed Culture System

Determination of the cell density within a packed bed culture system for the purposes of cell density control is usually made by extrapolation of a sample count taken from the centre of the bed during the culture process. However, this can lead to uncertainty about how accurately the growth rate of the cells is being recorded, as some regions of the bed may possess more of fewer cells than the sampled region. This is a problem with traditional packed bed culture. Another concern is that areas at the base of the vessel may not be receiving enough nutrients as the media enters the vessel through the inlet port in the vessel.

During two repeat runs under identical conditions of the perfusion system of the present invention the beds were sampled according to the locations outlined in FIG. 11 where nutrient media entered the base of the vessel through a single 8 mm diameter port in the middle of the vessel. FIG. 12 shows the cell distribution at the various locations indicated at Day 2, Day 6 and Day 10.

### Example 3

### Enhanced Cell Growth when Compared to Other Culture Methods

In the following experiment, a surface area addition rate of 40% substrate per day was used. This had a dramatic effect on cell doubling rate and decreased the time taken for the expansion process in the vessel from 12 to eight days when compared with tissue flask culture.

A smaller number of starting rings where used with and initial seeding number of 1.10×10⁶. As the experiment recovered 1.93×10⁸ cells from the vessel at the end of the run a 175 fold expansion was achieved. This is significantly higher than any reported fold expansion within a single vessel. Since the rate of surface addition appeared to keep pace with the cell population's growth rate a much shorter process time was made possible.

The cells maintained an exponential growth rate over an eight day period. The control (T175) flasks were passaged every 6 days with a flask sacrificed for cell counting at days 8 and 14 to provide more information for plotting comparative growth curves. The growth curves for both the control flasks and the perfused device are shown in FIG. 13.

The cumulative population doublings for both experiments are plotted from day six of the original growth curve profiling as this is where the experimental cell bank was established and it allows for comparison with the control flask populations.

The packed bed cultures and methods of the present invention provide a dramatic improvement over those described previously. For example, Mizukami et al (Biotechnol. Prog. 29(2) 568-572 (March-April 2013)) described the use of a disposable fixed bed culture system and achieved a fold expansion of only 7. Weber et al (Int. J. Artificial Organs, 33(8) 512-525 (2010)) using a fixed (non-expanding) bed design also only achieved an expansion ratio of 22.3. The present invention therefore allows expansion ratios which are significantly higher to be achieved.

To sum up, the present invention provides a novel packed bed cell cultivating system and associated method that could eliminate the limitation of conventional packed-bed bioreactors. The example method provided by the present invention could enhance a homogenized cell distribution in large scale packed-bed bioreactor and improve process efficiency significantly.

While the invention can be subject to various modifications and alternative forms, a specific example has been herein described in detail. It should be understood, however, that the invention is not to be limited to the particular form disclosed. The associated experimental results demonstrate the ability of this system and method to control process parameters by the controlled addition and subtraction of micro-substrates within this system.

1. A method of culturing cells in a cell culture device comprising a plurality of non-porous substrates in the form of a packed bed, a reservoir for cell culture media in fluid communication with the cell culture device, and a means for circulating the cell culture media from the reservoir through said device, in which the method comprises:
incubating the cells,
(ii) recirculating the media between the reservoir and the packed bed chamber,
(iii) introducing further substrates to the packed bed container once the cells have reached 40-50% confluence,
(iv) repeating step (iii) and adding additional substrates at a ratio of 20% to 60% extra surface area,
(v) optionally repeating step (iv),
(vi) comprising a step of mixing substrates in the device,
(vii) optionally sampling the cell culture to measure the cell density, and
(viii) recovering the cells.

2. The method of claim 1, further comprising sampling the cell culture to measure the cell density after recovering the cells.

3. The method of claim 1, wherein the number of cells present in the culture after introduction of further substrates in step (iii) and additional substrates in step (iv) is expanded at least 50-fold.

4. The method of claim 1, in which the substrates are mixed by physical agitation of the cell culture device.

5. The method of claim 1, in which the substrates are mixed by a magnetic field applied to the substrates.

6. The method of claim 1, in which the substrates are mixed by a pulse of liquid introduced into the cell culture device.

7. A cell-culture device comprising a packed bed chamber and a control unit, in which the control unit comprises a means for monitoring one or more process parameters, wherein the packed bed chamber comprises a plurality of openings for introducing or removing non-porous substrates and/or cells and permitting flow of cell culture media into and out of the device and wherein the device comprises a means for mixing the substrates.

8. The cell-culture device of claim 7, in which the device further comprises a media distribution manifold inside the packed bed chamber.

9. The cell-culture device of claim 7, in which the packed bed chamber is in fluid communication with a reservoir of cell culture media.

10. The cell-culture device of claim 7, in which the packed bed chamber is divided into separate compartments.

11. A system for culturing cells comprising the cell culture device of claim 7, a reservoir for cell culture media in fluid communication to the cell culture device, a means for circulating the cell culture media from the reservoir through said device, and a reservoir of cell culture non-porous substrates in connection to the cell culture device and a means for introducing said substrates from the reservoir to the cell culture device.

12. The system of claim 11, in which each substrate has a lumen with a circular cross section in which the diameter of the lumen is approximately the same as the vertical height of the substrate.

13. The system of claim 11, wherein said substrate comprises a tubular section of an inert material with a circular cross section, which has an interior lumen having a circular cross section in which the diameter of the lumen is approximately equal to half the diameter of the substrate, in which the vertical height of the substrate is approximately 1.5 times the diameter of the substrate and in which one end of the substrate has a channel cut perpendicular to the axis of symmetry of the lumen where said channel has a diameter approximately equal to the diameter of the lumen of the substrate and in which the substrate is further modified at the other end by the presence of one more channels.

14. The system of claim 11, wherein said substrate comprises a tubular section of an inert material with a circular cross section, which has an interior lumen having a circular cross section in which the diameter of the lumen is approximately equal to half the diameter of the substrate, in which the vertical height of the substrate is approximately 1.5 times the diameter of the substrate and in which a channel is cut perpendicular to the axis of symmetry of the lumen at the centre of the substrate where said channel has a diameter approximately equal to the diameter of the lumen of the substrate.

15. The system of claim 11, wherein said substrate comprises a tubular section of an inert material with a circular cross section, which has an interior lumen having a circular cross section in which the diameter of the lumen is approximately equal to half the diameter of the substrate, in which the vertical height of the substrate is approximately 1.5 times the diameter of the substrate and in which a channel is cut through the substrate at both ends of the substrate perpendicular to the axis of symmetry of the lumen where said channels have a diameter approximately equal to the diameter of the lumen of the substrate.

16. A non-porous cell culture substrate comprising a tubular section of an inert material with a circular cross section, which has an interior lumen having a circular cross section, in which the vertical height of the substrate is approximately 1.5 times the diameter of the substrate and in which the radius of the lumen is approximately equal to the thickness of the wall of the substrate surrounding the lumen.

17. The cell culture substrate of claim 16 in which the substrates are coated with a biologically active substance, for example a hormone, a growth factor, matrix protein or a mixture thereof.

18. The cell culture substrate of claim 16 in which the height of the substrate is from about 0.5 mm to about 10.0 mm.

19. The cell culture substrate of claim 16, in which the substrate comprises a plurality of lumina.
